(19) United States
(12) Patent Application Publication
(10) Pub. No.: US 2025/0276175 A1
(43) Pub. Date: Sep. 4, 2025

(54) FEEDBACK-BASED NEURAL ELECTRICAL STIMULATION SYSTEM

(71) Applicant: NATIONAL CHENG KUNG UNIVERSITY, Tainan (TW)

(72) Inventors: CHOU-CHING LIN, Tainan (TW); SHUENN-YUH LEE, Tainan (TW); ZHAN-XIAN LIAO, Tainan (TW); PENG-TING CHEN, Tainan (TW); PEI-JUNG WU, Tainan (TW); PI-SHAN SUNG, Tainan (TW); YA-WEN CHANG, Tainan (TW); GIN-SHIN CHEN, Tainan (TW); GIA-SHING SHIEH, Tainan (TW)

(21) Appl. No.: 18/975,040

(22) Filed: Dec. 10, 2024

Related U.S. Application Data
(60) Provisional application No. 63/559,311, filed on Feb. 29, 2024.

(51) Int. Cl. A61N 1/36; A61N 1/02; A61N 1/04

(52) U.S. Cl. CPC A61N 1/36031; A61N 1/025; A61N 1/0456; A61N 1/0472; A61N 1/36025; A61N 1/36036

(57) ABSTRACT

The present disclosure provides a feedback-based neural electrical stimulation system including a wearable feedback-based neural electrical stimulation device. The feedback-based neural electrical stimulation device includes: a sensing module, for measuring physiological feedback signals from a user, including a photoplethysmography (PPG) sensor and an electrodermal activity (EDA) sensor; an electrical stimulator, for providing electrical stimulation to the user, including a tragus electrode and a concentric electrode; and a digital controller electrically connected to both the sensing module and the electrical stimulator, for receiving the physiological feedback signals from the sensing module and outputting electrical stimulation control signals to the electrical stimulator so as to control the electrical stimulation.

### FIELD

The present disclosure relates to a feedback-based neural electrical stimulation system, and more particularly to a feedback-based neural electrical stimulation system for neuromodulation.

### BACKGROUND

While current neurostimulation technologies and devices have achieved significant progress in addressing various neurological disorders, they continue to face numerous limitations and challenges. These devices are predominantly used to treat conditions, such as chronic pain, depression, and epilepsy, but their therapeutic efficacies remain open to improvement. Most neurostimulation devices in use today operate with fixed electrical stimulation parameters, including duration, frequency, and intensity. This rigid approach fails to adapt to the unique physiological needs of individual patients, making it challenging to optimize treatment outcomes. For example, traditional vagus nerve stimulation (VNS) devices often necessitate invasive surgical procedures, which not only heighten patient risk but also introduce inconvenience, thus discouraging many potential receivers for treatments.

More notably, most neurostimulation devices on the market today lack real-time physiological feedback mechanisms. This limitation prevents the devices from adjusting stimulation parameters dynamically based on the user's current physiological state, thus significantly hindering treatment personalization and overall efficiency. In an era increasingly focused on precision medicine, this “one-size-fits-all” approach is clearly inadequate for meeting the demands of modern healthcare. The absence of physiological feedback not only compromises treatment effectiveness but also risks missing the optimal treatment window, as the devices are unable to promptly respond to changes in patient conditions.

Furthermore, from the perspective of user experience, many existing neurostimulation devices leave considerable room for improvement in terms of convenience and comfort. Specifically, some devices are impractical for long-term wear or daily use due to their bulky design or complex operation, which not only impacts patients' quality of life but also diminishes treatment adherence. Ideally, neurostimulation devices should be lightweight, easy to use, comfortable, and integrating seamlessly into users' daily routines rather than imposing an additional burden.

### SUMMARY

In a first aspect of the present disclosure, a feedback-based neural electrical stimulation system including a wearable feedback-based neural electrical stimulation device is provided. The feedback-based neural electrical stimulation device may include: a sensing module, for measuring physiological feedback signals from a user, including: a photoplethysmography (PPG) sensor for measuring PPG signals; and an electrodermal activity (EDA) sensor for measuring EDA signals; an electrical stimulator, for providing electrical stimulation to the user, including: a tragus electrode configured to attach to the user's tragus for providing transcutaneous auricular vagus nerve stimulation (taVNS); and a concentric electrode including a center electrode and a ring electrode. The concentric electrode configured to attach to the user's skin for providing transcranial direct current stimulation (tDCS); and a digital controller electrically connected to both the sensing module and the electrical stimulator for receiving the physiological feedback signals from the sensing module and outputting electrical stimulation control signals to the electrical stimulator so as to control the electrical stimulation.

According to a first aspect of the present disclosure, the digital controller may receive the PPG signals from the sensing module and convert the PPG signals to frequency domain for obtaining heart rate variability, analyze high-frequency components and low-frequency components of the heart rate variability, determine an activity level of a parasympathetic nerve based on an energy ratio between the high-frequency components and the low-frequency components, and adjust the electrical stimulation control signals accordingly to the control of the electrical stimulation of the electrical stimulator.

According to a first aspect of the present disclosure, the high-frequency components may be in a frequency range of 0.15 to 0.40 Hz, and the low-frequency components may be in a frequency range of 0.06 to 0.15 Hz.

According to a first aspect of the present disclosure, the digital controller may receive the EDA signals from the sensing module and decompose the EDA signals into a skin conductance level (SCL) component and a skin conductance response (SCR) component, determine an activity level of a sympathetic nerve based on the SCL component, and adjust the electrical stimulation control signals accordingly to the control of the electrical stimulation of the electrical stimulator.

According to a first aspect of the present disclosure, the digital controller may determine an activity level of a parasympathetic nerve based on the PPG signals and determine an activity level of a sympathetic nerve based on the EDA signals, and adjust the electrical stimulation control signals to control a stimulation frequency and a pulse width of the electrical stimulation based on the activity level of the parasympathetic nerve and the activity level of the sympathetic nerve.

According to a first aspect of the present disclosure, the feedback-based neural electrical stimulation system may further include: a mobile control device, for wireless communication with the wearable feedback-based neural electrical stimulation device, configured to display the physiological feedback signals and the electrical stimulation control signals and provide control commands to adjust the electrical stimulation control signals; and a server device, for wireless communication with the wearable feedback-based neural electrical stimulation device and the mobile control device, configured to store and analyze the physiological feedback signals and the electrical stimulation control signals.

According to a first aspect of the present disclosure, the wearable feedback-based neural electrical stimulation device may further include: a wireless transmission module, for wireless communication with the mobile control device and the server device, electrically connected to the digital controller to transmit the physiological feedback signals, the electrical stimulation control signals, and the control commands.

According to a first aspect of the present disclosure, the wearable feedback-based neural electrical stimulation device may further include: a body configured to accommodate the digital controller with the tragus electrode and the concentric electrode disposed on a surface of the body; an earplug element coupled to the body for securing the wearable feedback-based neural electrical stimulation device to the user's ear; and a sensing element disposed on a surface of the earplug element to contact the user's ear and configured to accommodate the PPG sensor and the EDA sensor.

According to a first aspect of the present disclosure, the tragus electrode may be a contact electrode or a snap-fit electrode.

According to a first aspect of the present disclosure, the contact electrode may be a direct contact electrode or a spring-mechanism electrode.

According to a first aspect of the present disclosure, the snap-fit electrode may include a male snap and a female snap that are detachably assembled with each other, one of the male snap and the female snap may contact the user's ear, and the other may be disposed on the body.

### BRIEF DESCRIPTION OF THE DRAWINGS

When read in conjunction with the accompanying drawings, the following detailed description will provide a better understanding of the present disclosure, in which:

FIG. 1 is a system architecture diagram of a feedback-based neural electrical stimulation system according to an implementation of the present disclosure.

FIG. 2 is a basic architecture diagram showing a relationship between elements in the feedback-based neural electrical stimulation system of the present disclosure.

FIG. 3 is a schematic diagram showing a feedback-based neural electrical stimulation device worn on a user's ear according to an implementation of the present disclosure.

FIG. 4 is a perspective view of the feedback-based neural electrical stimulation device of the present disclosure.

FIG. 5A is a schematic diagram of a direct contact tragus electrode according to an implementation of the present disclosure.

FIG. 5B is a schematic diagram of a spring-mechanism tragus electrode according to another implementation of the present disclosure.

FIG. 6A is a schematic diagram of a directly connected snap-fit tragus electrode according to an implementation of the present disclosure.

FIG. 6B is a schematic diagram of an extended connected snap-fit tragus electrode according to another implementation of the present disclosure.

FIG. 7 is a schematic diagram of a concentric electrode according to an implementation of the present disclosure.

FIG. 8 is a schematic diagram of a body of the feedback-based neural electrical stimulation device according to an implementation of the present disclosure.

### DETAILED DESCRIPTION

The following description contains specific information related to exemplary implementations of the present disclosure. The drawings of the present disclosure and their accompanying detailed descriptions are exemplary implementations only. However, the present disclosure is not limited to these exemplary implementations. One with ordinary skill in the art will envision other variations and implementations of the present disclosure. Unless otherwise specified, same or corresponding elements in the drawings may be indicated by same or corresponding reference numerals. Furthermore, the drawings and illustrations in the present disclosure are generally not drawn to scale and are not intended to correspond to actual relative dimensions.

For consistency and ease of understanding, similar features are designated by reference numerals in the exemplary drawings (although not so designated in some examples). However, features in different implementations may differ in other aspects, and therefore, should not be narrowly limited to the features shown in the drawings.

The terms “at least one implementation,” “an implementation,” “multiple implementations,” “different implementations,” “some implementations,” “the present implementation,” and the like indicate that implementations so described may include particular features, structures, or characteristics, but not every possible implementation of the present disclosure must include the particular features, structures, or characteristics. Moreover, repeated use of the phrases “in an implementation” or “in the present implementation” does not necessarily refer to the same implementation, although they may. Furthermore, when a phrase such as “implementation” is used in association with “the present disclosure,” it does not mean all implementations of the present disclosure must include specific features, structures, or characteristics, and should be understood as “at least some implementations of the present disclosure” include the stated features, structures, or characteristics.

The term “coupled” is defined as connected, whether directly or indirectly through intervening elements, and is not necessarily limited to physical connections or used to describe a particular sequence. When the terms “include,” “comprising,” or any variations thereof are used, they are intended to cover a non-exclusive inclusion, namely, “including but not limited to,” which explicitly indicates an open-ended inclusion or relationship of combinations, groups, series and equivalents. For example, a process, method, system, product, or apparatus including a series of steps or modules is not limited to those listed steps or modules, but may optionally include unlisted steps or modules, or may optionally include other steps or modules inherent to such processes, methods, products, or apparatus.

Additionally, specific details such as functional entities, techniques, protocols, and standards are described for purposes of explanation and not limitation, to provide an understanding of the described technology. In other examples, detailed descriptions of well-known methods, techniques, systems, architectures, and the like are omitted to avoid obscuring the description with unnecessary detail.

The terms “first,” “second,” “third,” and so forth in the specification and the above drawings are used to distinguish different objects rather than to describe a particular sequence.

The following provides a further detailed description of the present disclosure in conjunction with the drawings and implementations.

The present disclosure fundamentally relates to a feedback-based neural electrical stimulation device, system, and use thereof, for integrating dual-mode neuromodulation of transcutaneous auricular vagus nerve stimulation (taVNS) and transcranial direct current stimulation (tDCS). The device, system, and the use thereof provide more effective treatment solutions for different types of neurological disorders, such as for the auxiliary treatment of acute and subacute stroke patients, by providing more effective neural function reconstruction through real-time physiological feedback mechanisms.

The feedback-based neural electrical stimulation device, system, and use thereof described in the present disclosure may be used by a user to assist in medical procedures for humans or mammals (e.g., another patient). In some implementations, the user may be an individual using the feedback-based neural electrical stimulation device, system, and use thereof of the present disclosure. In some implementations, the patient may be another individual who is the subject of medical procedures performed using the feedback-based neural electrical stimulation device, system, and use thereof of the present disclosure.

Referring to FIG. 1, which is a system architecture diagram of a feedback-based neural electrical stimulation system 100 according to an implementation of the present disclosure. As shown in the figure, the feedback-based neural electrical stimulation system 100 may include a feedback-based neural electrical stimulation device 1, a mobile control device 2, and a server device 3.

In some implementations, the feedback-based neural electrical stimulation device 1 may be a device worn by a user, which may include one or more sensors for detecting physiological signals, such as heart rate, brain waves, electrodermal activity signals, electromyographic signals, or neural signals, and may include one or more stimulators to provide corresponding neural electrical stimulation to the user, while the user may also manually adjust the settings of the feedback-based neural electrical stimulation device 1 according to his/her needs, or automatic adjustments through artificial intelligence analysis may be made.

In some implementations, the feedback-based neural electrical stimulation device 1 may utilize an integrated digital controller to perform efficient edge computing for on-chip signal processing and control, or provide intelligent platform analysis through the mobile control device, which enables the feedback-based neural electrical stimulation device 1 to perform real-time processing of physiological signals from sensors, thus generating precise control signals to regulate the electrical stimulation output of the stimulator.

In some implementations, the mobile control device 2 may be an electronic device, such as a smartphone, digital assistant, or computer, and users may set electrical stimulation parameters for the feedback-based neural electrical stimulation device 1 through the mobile control device 2, as well as performing artificial intelligence analysis. The mobile control device 2 may also receive and display information transmitted from the feedback-based neural electrical stimulation device 1, which may include detection results, provided electrical stimulation modes, and artificial intelligence analysis results.

In some implementations, the mobile control device 2 may perform artificial intelligence analysis through a dedicated application designed for the feedback-based neural electrical stimulation system 100, thus allowing users to track and record the effects and responses of each electrical stimulation session. This data processed by the mobile control device 2 not only helps users find the most suitable electrical stimulation mode but may also provide real-time feedback and suggestions through a user-friendly interface, thus enhancing user experience and stimulation personalization.

In some implementations, the server device 3 may be a repository, such as a cloud device for storing multiple information, to collect information from the feedback-based neural electrical stimulation device 1 transmitted via the mobile control device 2, and perform signal processing and artificial intelligence analysis on this information in order to modify relevant settings in the feedback-based neural electrical stimulation device 1. Then these modifications may be fed back to the feedback-based neural electrical stimulation device 1 either directly or through the mobile control device 2 to achieve optimization of electrical stimulation therapy.

In some implementations, the server device 3 may serve as a data repository to collect and store electrical stimulation data from all users. Through analysis and processing of this large amount of data, as well as using de-identification techniques to protect user privacy, the server device 3 may extract valuable demographic information. This information helps develop and optimize artificial intelligence models that are specifically targeted at certain regions or populations to provide users with more accurate and personalized electrical stimulation treatment plans, while also promoting innovation and advancement in the widespread application of electrical stimulation technology across various populations.

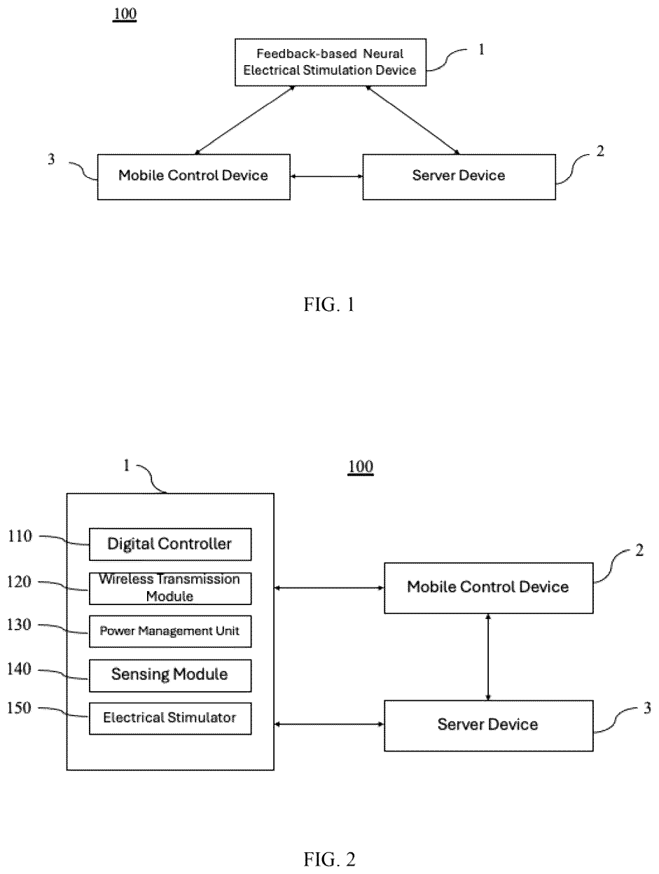

For a more detailed explanation of the internal architecture of each device, please refer to FIG. 2, which is a basic architecture diagram showing a relationship between elements in the feedback-based neural electrical stimulation system 100 of the present disclosure. The feedback-based neural electrical stimulation system 100 may include a feedback-based neural electrical stimulation device 1, a mobile control device 2, and a server device 3. The following will describe the constituent elements and their functions for each device.

In some implementations, the feedback-based neural electrical stimulation device 1 may include a digital controller 110, a wireless transmission module 120, a power management unit 130, a sensing module 140, and an electrical stimulator 150. In some implementations, the digital controller 110 may have predetermined computing capability and register for receiving commands that are transmitted from the mobile control device 2 via the wireless transmission module 120. The commands may include parameters controlling the output of the electrical stimulator 150, such as anodic stimulation intensity, cathodic stimulation intensity, anodic stimulation time, cathodic stimulation time, interval time, stimulation cycle, and total stimulation count, whose specific values may be set and adjusted according to electrical stimulation therapy requirements.

In some implementations, the wireless transmission module 120 may employ wireless transmission technologies, such as WiFi or Bluetooth, and different wireless transmission modules may be selected based on factors, such as usage scenario, treatment procedure time, and sensing data volume.

In some implementations, the power management unit 130 may include a charging interface, a rechargeable battery, a voltage regulator, and a voltage booster, where the rechargeable battery may be charged via the charging interface, while the voltage regulator may be used to convert battery voltage to system required voltage, and the voltage booster may be used to boost system voltage to the voltage level that is required by the electrical stimulator 150.

In some implementations, the sensing module 140 may be used to measure physiological signals and convert them into electrical signals for use by the digital controller 110, and the sensing data may be transmitted to the mobile control device 2 by the digital controller 110 through the wireless transmission module 120. The physiological signals may be feedback related to electrical stimulation therapy, including photoplethysmography signals and electrodermal signals, which will be explained in more detail later.

The following will use an ear-worn feedback-based neural electrical stimulation device as an example to provide a more detailed description of the feedback-based neural electrical stimulation device, system, and use thereof of the present disclosure, in conjunction with the implementations shown in the figures.

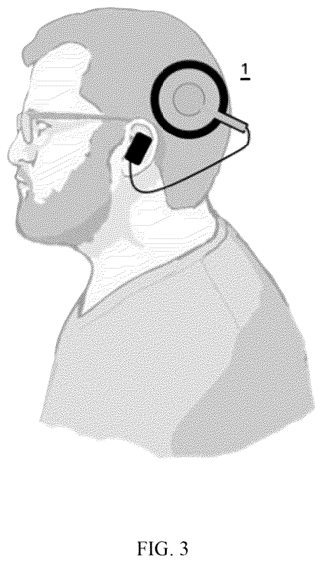

In some implementations, the ear-worn feedback-based neural electrical stimulation device may have structures, such as ear-hook or earplug for securing the feedback-based neural electrical stimulation device to the user's ear. Refer to FIG. 3, which is a schematic diagram showing a feedback-based neural electrical stimulation device worn on a user's ear according to an implementation of the present disclosure. As shown in the figure, the feedback-based neural electrical stimulation device of the present disclosure may be worn on the user's ear with an earplug structure, and includes electrodes for providing electrical stimulation to the vagus nerve branches in the tragus, and additionally integrates a concentric electrode for providing transcranial direct current stimulation. In a preferred implementation, the feedback-based neural electrical stimulation device of the present disclosure may be worn on the user's left ear with an earplug structure.

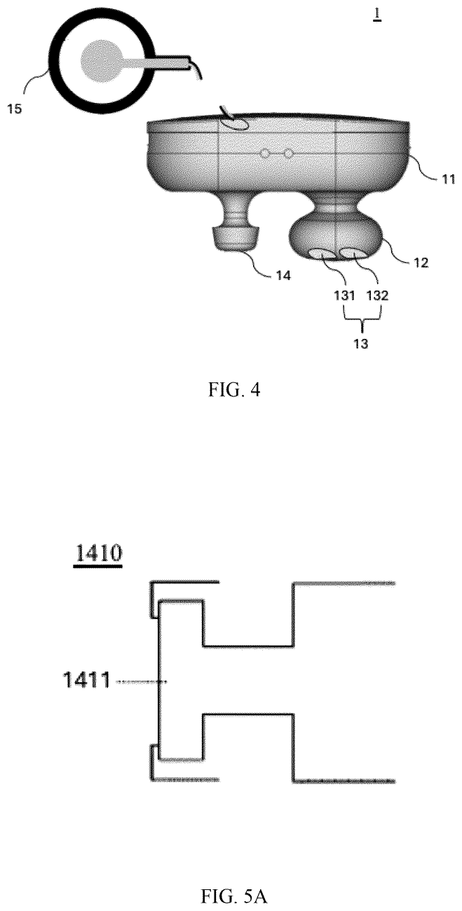

Refer to FIG. 4, which is a perspective view of the feedback-based neural electrical stimulation device 1 of the present disclosure. The feedback-based neural electrical stimulation device 1 may include a body 11, an earplug element 12, a sensing element 13, a tragus electrode 14, and a concentric electrode 15. The sensing element 13 may be a part of the sensing module 140, and the tragus electrode 14 and the concentric electrode 15 may be parts of the electrical stimulator 150. The following will provide a detailed description of the structural features and functions of the tragus electrode 13 and sensing element 14.

In some implementations, the sensing element 13 may include a sensor 131 for detecting photoplethysmography (PPG) and a sensor 132 for detecting electrodermal activity (EDA). These sensors 131, 132 are disposed on the surface of the earplug element 12 to contact the skin of the user, and after capturing physiological signals, the physiological signals first undergo signal processing, such as pre-amplification and filtering, through analog front-end circuits before being transmitted to the digital controller 110 for subsequent processing. In some implementations, the digital controller 110 may include a driving module for controlling the output power of the PPG sensor 131 and generating voltage signals for the EDA sensor 132.

In some implementations, the PPG sensor 131 may include a light emitting diode (LED) and a light receiving component. The LED may be used to emit lights of specific wavelengths The power of the LED may be controlled by the driving module in the digital controller 110, and these lights experience different degrees of attenuation when passing through skin tissue and blood vessels with time-varying diameters, with the attenuated reflected lights being detected by the light receiving component. The driving LED and light receiving component are fixed on the surface of the earplug element 12 and may have a predetermined distance between them to ensure optimal sensing effect.

In some implementations, the EDA sensor 132 may include a driving electrode and a capturing electrode. The driving electrode outputs voltage signals from the driving module 1105 in the digital controller 110, and after these voltage signals pass through skin tissue impedance of the user, the current signals are captured by the capturing electrode. The driving electrode and capturing electrode may employ direct contact or spring mechanism designs to ensure tight contact with the skin tissue of the user.

In some implementations, the tragus electrode 14 may be configured as a contact electrode. Specifically, the tragus electrode 14 may adopt a direct contact design that allows the electrode to directly contact the skin surface, which is particularly suitable for situations requiring precise electrical stimulation. In some implementations, the tragus electrode 14 may also adopt a design with spring mechanism to ensure the electrode could more tightly and evenly conform to the stimulation site, which is particularly suitable for electrical stimulation on dynamic or irregular surfaces.

In some implementations, the tragus electrode 14 may be made of medical-grade stainless steel to ensure its durability and safety. During use, conductive gel may be applied to the surface of the tragus electrode 14 to enhance electrical connection with the user's skin and enable the tragus electrode 14 to firmly adhere to the skin surface.

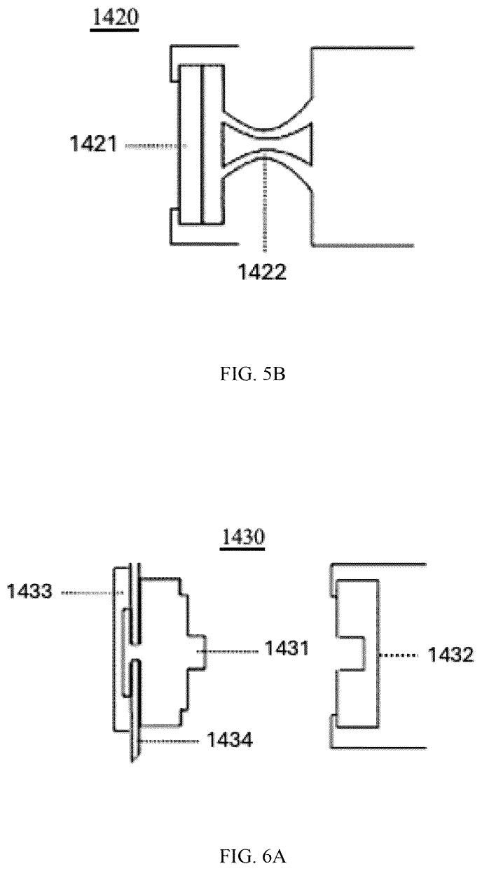

Refer to FIG. 5A, which is a schematic diagram of a direct contact tragus electrode according to an implementation of the present disclosure. In some implementations, the direct contact tragus electrode 1410 may be made of a medical-grade metal conductor 1411, follow electrical stimulation charge density guidelines, and have a contact surface area corresponding to its maximum stimulation intensity. The direct contact tragus electrode 1410 may be fixed on the surface of the feedback-based neural electrical stimulation device 1, at a designated position, to conform to the target skin tissue of the user, and may be connected to the circuit stimulation output port inside the body 11 through wires.

Refer to FIG. 5B, which is a schematic diagram of a spring-mechanism tragus electrode according to another implementation of the present disclosure. In some implementations, the spring-mechanism tragus electrode 1420 may also be made of a medical-grade metal conductor 1421, similarly follow electrical stimulation charge density guidelines, and have a contact surface area corresponding to its maximum stimulation intensity. The spring-mechanism tragus electrode 1420 may be disposed on the surface at a designated position of the feedback-based neural electrical stimulation device 1 in a floatable state. The spring-mechanism tragus electrode 1420 may, through the internal spring 1422 of the device, use the pressure from the extension and contraction of the spring 1422 to conform to the target skin tissue of the user, and connect to the circuit stimulation output port inside the body 11 through wires.

In some implementations, the tragus electrode 14 may also be configured as a snap-fit electrode, which may be configured to directly connect the feedback-based neural electrical stimulation device 1, or may be configured to connect through a connector extending from the feedback-based neural electrical stimulation device 1, which is suitable for situations requiring precise electrical stimulation. The tragus electrode 14 may be adjusted according to treatment needs to ensure maximum therapeutic effect.

In some implementations, the snap-fit tragus electrode may be made of high-performance conductive materials, such as silver or silver chloride, to ensure good electrochemical stability and low skin resistance of the electrode, thus effectively promoting electrical stimulation conduction. In some implementations, the snap-fit tragus electrode may also use gel or gel-like substances containing electrolytes to significantly enhance the contact between the electrode and skin, thus effectively reducing contact resistance, increasing conductivity, and ensuring effective delivery of electrical stimulation.

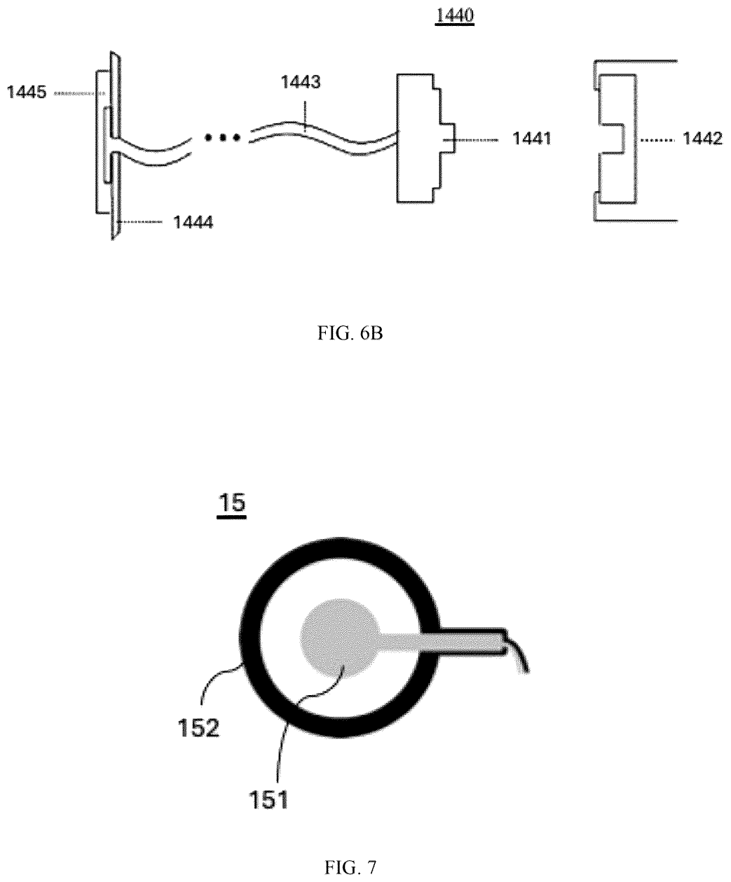

Refer to FIG. 6A, which is a schematic diagram of a directly connected snap-fit tragus electrode according to an implementation of the present disclosure. In some implementations, the snap-fit tragus electrode 1430 may be made of a medical-grade metal conductor and divided into a male snap 1431 and a female snap 1432. The male snap 1431 may have an electrode 1434 for contacting the user, follow electrical stimulation charge density guidelines, and have a contact surface area corresponding to its maximum stimulation intensity. The electrode 1434 may include a conductive part 1433 attached with conductive gel or gel-like substances. Furthermore, the male snap 1431 and female snap 1432 may be directly snapped together, fixed on the surface of the feedback-based neural electrical stimulation device 1, at a designated position, and connected to the circuit stimulation output port inside the body 11 through wires. In some implementations, the installation positions of the male snap 1431 and female snap 1432 may be interchangeable, that is, the male snap may be disposed on the body while the female snap contacts the user to achieve the same snap-fit effect.

Refer to FIG. 6B, which is a schematic diagram of an extended connected snap-fit tragus electrode according to another implementation of the present disclosure. In some implementations, the snap-fit tragus electrode 1440 may be made of a medical-grade metal conductor and divided into a male snap 1441 and a female snap 1442. Additionally, the snap-fit tragus electrode 1440 may include an interconnected wire 1443 and an electrode 1444. The electrode 1444 may follow electrical stimulation charge density guidelines and have a contact surface area corresponding to its maximum stimulation intensity. The electrode 1444 may include a conductive part 1445, and the conductive part 1445 may be attached with conductive gel or gel-like substances. The electrode 1444 may be connected to either the male snap 1441 or female snap 1442 through the wire 1443, while the other of the male snap 1441 and female snap 1442 may be fixed on the surface of the feedback-based neural electrical stimulation device 1, at a designated position, and may be connected to the circuit stimulation output port inside the body 11 through the wire 1443.

In some implementations, refer to FIG. 7, which is a perspective view of a concentric electrode 15 according to an implementation of the present disclosure. The concentric electrode 15 may include a center electrode 151 and a ring electrode 152, where both the center electrode 151 and ring electrode 152 may be made of a medical-grade metal conductor, follow electrical stimulation charge density guidelines, and have contact surface areas corresponding to their maximum stimulation intensity. The center electrode 151 may be disposed at the center position of the ring electrode 152, and there may be a predetermined gap distance between the center electrode 151 and ring electrode 152. In some implementations, the gap distance between the center electrode 151 and ring electrode 152 may be 1-3 centimeters, preferably 2 centimeters.

In some implementations, the center electrode 151 and ring electrode 152 may separately connect to the circuit stimulation output port inside the body 11 through wires to receive different electrical stimulation signals. The center electrode 151 may serve as the anode while the ring electrode 152 may serve as the cathode to produce precise transcranial direct current stimulation effects. In some implementations, the center electrode 151 and ring electrode 152 may also use gel or gel-like substances containing electrolytes to significantly enhance contact with the skin, thus effectively reducing contact resistance and increasing conductivity.

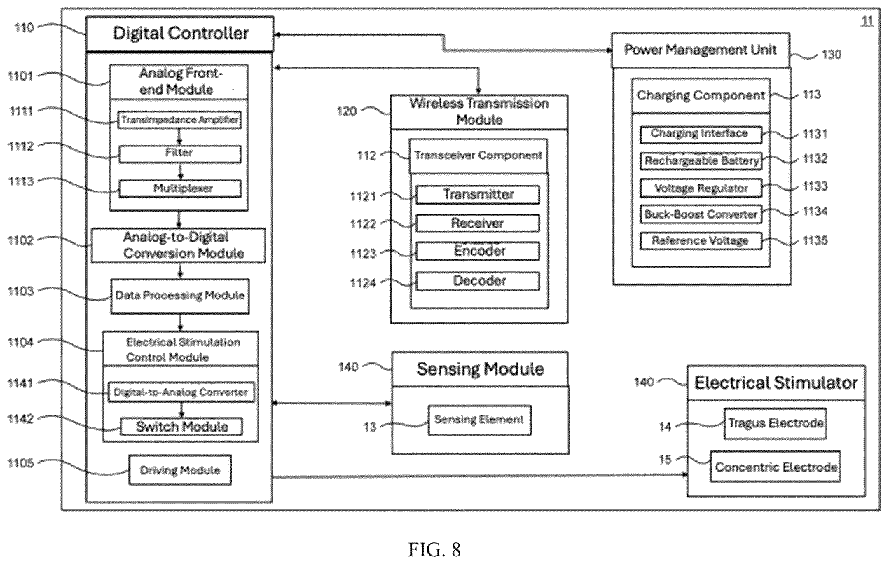

Refer to FIG. 8, which is a schematic diagram of a body of the feedback-based neural electrical stimulation device according to an implementation of the present disclosure. In some implementations, the body 11 may be powered by a rechargeable battery and may include the digital controller 110, a transceiver component 112, and a charging component 113. The transceiver component 112 may be part of the wireless transmission module 120, and the charging component 113 may be part of the power management unit 130. In some implementations, the digital controller 110 may be used to provide control signals to the tragus electrode 14 and the concentric electrode 15, and process signals obtained from the sensing element 13 to regulate the control signals.

In some implementations, the digital controller 110 in the body 11 of the feedback-based neural electrical stimulation device may include an analog front-end module 1101, an analog-to-digital conversion module 1102, a data processing module 1103, an electrical stimulation control module 1104, and a driving module 1105. The analog front-end module 1101 may include a transimpedance amplifier 1111, a filter 1112, and a multiplexer 1113. The analog front-end module 1101 may be used to process PPG signals (e.g., measuring photodiode output current) and EDA physiological signals (e.g., measuring current applied to skin impedance). The driving module 1105 may be used to control the LED output power of the PPG sensor 131 and generate voltage signals required by the EDA sensor 132. In some implementations, the driving module 1105 may include LEDs of specific wavelengths required for PPG signals and their LED drivers, and may include voltage outputters required for EDA signals.

In some implementations, the startup operation of the analog front-end module 1101 may be powered by stable voltage supply provided by the charging component 113. The PPG signal involves measuring photodiode output current, while the EDA physiological signal involves measuring current applied to skin impedance. The transimpedance amplifier 1111 may be used to convert current to voltage, and since physiological signal feedback is often tiny signals that do not match the voltage range for backend processing, the transimpedance amplifier 1111 also has provide amplification functionality. The filter 1112 may, after being electrically connected the transimpedance amplifier 1111, be used for filtering physiological signals and remove noises that are outside the required frequency band, such as wireless and circuit board internal noise coupling, as well as motion artifacts and common-mode signals that may be generated by human body movements. The multiplexer 1113 may, after being electrically connected to the filter 1112, be used to process PPG signals and EDA signals through methods, such as time division or frequency division, thus allowing signals to share the backend analog-to-digital conversion module 1102.

In some implementations, the analog-to-digital conversion module 1102 may be used to convert signals that are processed by the analog front-end module 1101 into digital encoding for further processing by the data processing module 1103. The data processing module 1103 may include digital signal processing circuits for processing and collecting PPG and EDA physiological signals from the user during electrical stimulation.

In some implementations, the data processing module 1103 may further include a computation module for processing PPG and EDA signals. In some implementations, the computation module may be specifically designed for neural function monitoring requirements of stroke patients to provide real-time and precise autonomic nervous function assessment, and accordingly provide feedback to adjust stimulation parameters.

In some implementations, the computation module may convert PPG signals, through frequency domain analysis, to obtain heart rate variability, and then determine the activation degree of the parasympathetic component in the autonomic nervous system from the heart rate variability. Specifically, when processing PPG signals, the computation module may first convert the PPG signals to the frequency domain, then calculate the energy ratio between a high frequency (0.15-0.40 Hz) and a low frequency (0.06-0.15 Hz) of heart rate variability, where high frequency components may represent parasympathetic nerve activity in the autonomic nervous system, while low frequency components may reflect the combined activity of sympathetic and parasympathetic nerves. In some implementations, for stroke patients' needs, the computation module may additionally analyze time domain indicators of heart rate variability, including the standard deviation of adjacent heartbeat intervals and the root mean square of differences between adjacent heartbeat intervals. Furthermore, the computation module may also analyze nonlinear indicators, such as approximate entropy and sample entropy, to more comprehensively evaluate the patient's autonomic nervous function recovery status. The above analytical functions of the computation module may also be implemented through the artificial intelligence platform of the mobile control device 2.

In some implementations, the data processing module 1103 may capture EDA signals through the EDA sensor, then use a rolling ball algorithm to analyze the detected EDA signals. Specifically, the rolling ball algorithm may dynamically adjust the radius and scale of the sphere based on signal characteristics, and using rolling ball computations of different scales to decompose and process signals, as well as to decompose the signal into SCL (skin conductance level) and SCR (skin conductance response) components, where the SCL signal will serve as an indicator of sympathetic nerve component activation in the autonomic nervous system. A smaller diameter ball may be used for preliminary noise filtering, and a larger diameter ball may be used to separate the slowly varying SCL component. For example, a ball with a 0.25-second sphere size may be used for preliminary noise filtering, and a ball with a 2.5-second sphere size may be used for signal decomposition to separate the slowly varying SCL component. In some implementations, for stroke patients, the computation module may use SCL as the main indicator for evaluating sympathetic nerve component activation, while analyzing SCR frequency and amplitude characteristics, and establishing short-term trend charts to evaluate patients' immediate response to treatment. The above analytical functions of the data processing module may also be implemented through the artificial intelligence platform of the mobile control device 2.

In some implementations, the computation module may establish a comprehensive scoring system to determine the user's autonomic nervous function status. When the high-to-low frequency ratio is between 1.5 and 2.0, and the SCL fluctuation amplitude is within ±20% of the baseline, it may be considered a normal state. When the high-to-low frequency ratio drops to between 1.0 and 1.5, or the SCL fluctuation amplitude exceeds ±20%, but is not reaching 40% of the baseline, it may be determined as mild abnormality. When the high-to-low frequency ratio drops to between 0.5 and 1.0, or the SCL fluctuation amplitude exceeds ±40%, but is not reaching 60% of the baseline, it may be determined as moderate abnormality. When the high-to-low frequency ratio is below 0.5, or the SCL fluctuation amplitude exceeds ±60% of the baseline, it may be determined as severe abnormality. The computation module may transmit these analytical results in real-time to the electrical stimulation control module 1104, as a basis for adjusting stimulation parameters to provide more precise neuromodulation therapy. The above analytical functions of the computation module may also be implemented through the artificial intelligence platform of the mobile control device 2, and exchange information with the digital controller 110, via the wireless transmission module 120, to achieve the purpose of adjusting stimulation parameters.

In some implementations, the data processing module 1103 may transmit the analytical results of both signals, namely the activation information representing sympathetic and parasympathetic nerve components, respectively, to the electrical stimulation control module 1104. Under normal circumstances, one of these two incoming signals may be selected for use, and if both signals are received simultaneously, the PPG signal takes priority.

In some implementations, the digital controller 110 may adopt different control strategies based on different physiological signal characteristics. For photoplethysmography signals, the digital controller 110 may use standard proportional-derivative control, where the proportional term may be adjusted based on the deviation between current high-to-low frequency ratio and target value, while the derivative term may reflect the rate of change of this deviation, with both collectively determining the adjustment amount of taVNS stimulation parameters. Specifically, when the deviation is positive and shows an increasing trend, the controller may correspondingly increase stimulation intensity. When the deviation is negative and shows a decreasing trend, the controller may reduce stimulation intensity.

In some implementations, for electrodermal signals, the digital controller 110 may adopt a fuzzy control strategy, fuzzifying the SCL value and rate of change into multiple levels (e.g., low, medium, high), and determine output based on preset fuzzy rules. For example, when SCL is at a “high” level and the rate of change is “positive”, it may trigger rules to reduce stimulation intensity. When SCL is at a “low” level and the rate of change is “negative”, it may trigger rules to increase stimulation intensity.

In some implementations, the electrical stimulator 150 may output multiple waveforms, including square waves, triangular waves, trapezoidal waves, etc. The square waves may be set as the default waveform, mainly considering its better neural stimulation effect and simple implementation characteristics. There are safety limits for electrical stimulation intensity that are maintained below 4 mA, and the stimulation time for a single treatment does not exceed 30 minutes. In some implementations, taking stroke patients as an example, and during the acute phase of stroke (e.g., within 2 weeks after onset), the pulse width may be set to 0.2-0.5 ms with a frequency range of 20-100 Hz, while during the subacute phase (e.g., 2 weeks to 3 months after onset), the pulse width may be adjusted to 0.5-1 ms with a frequency range expanded to 20-250 Hz.

In some implementations, the electrical stimulation control module 1104 may include a digital-to-analog converter 1141 and a switch module 1142. The digital-to-analog converter 1141 may be used to receive commands and parameters from the digital controller 110, and convert digital signals to analog voltage in order to control output waveform amplitude (e.g., square wave intensity). The switch module 1142 may contain multiple electronic switches, and may be used, after being electrically connected to the digital-to-analog converter 1141, to control the phase and temporal characteristics of stimulation waveforms. Through precise control of these switches' opening and closing timing, the switch module 1142 may implement different stimulation modes. In monophasic stimulation mode, the switch module 1142 may control anodic current flow time and charge release time. While in biphasic stimulation mode, the switch module 1142 may control the timing of anodic current flow, cathodic current flow, and charge release. Furthermore, the switch module 1142 may also dynamically adjust waveform duty cycle according to treatment needs in order to optimize stimulation effects.

In some implementations, the electrical stimulation control module 1104 may use standard proportional-derivative control or fuzzy control to adjust electrical stimulation signals. Specifically, the electrical stimulation control module 1104 may adjust aVNS electrical stimulation signal intensity based on received activation information of sympathetic and parasympathetic nerve components. For example, relatively higher frequencies (20-250 Hz) and short pulses tend to excite relatively thicker myelinated A fibers, thus activating the parasympathetic nervous system, while relatively lower frequencies (0.5-10 Hz) and elongated pulses tend to activate both relatively thick and thin fibers, thus activating the sympathetic nervous system. The electrical stimulation control module 1104 may utilize this antagonistic mechanism between sympathetic and parasympathetic nerves for control.

In some implementations, when using both transcutaneous auricular vagus nerve stimulation (taVNS) and transcranial direct current stimulation (tDCS) simultaneously, the electrical stimulation control module 1104 sequentially performs taVNS first, followed by tDCS after completion of the taVNS, to achieve an optimal therapeutic effect. Specifically, when performing a taVNS stimulation, the electrical stimulation control module 1104 controls the switch module 1142 to divide its output current path into three states: when current flows from the digital-to-analog converter 1141, through the anode electrode, and to the cathode electrode, the current path is anodic stimulation; when the current direction is reversed, i.e., flowing from the cathode electrode to the anode electrode, the current path is cathodic stimulation; and when both electrodes are simultaneously connected to system ground, the current path is the charge release state. When performing a tDCS stimulation, the digital-to-analog converter 1141 in the electrical stimulation control module 1104 generates a current waveform with an initial ramp-up slope and a final ramp-down slope, thus maintaining stable direct current intensity output during the period, with a stimulation intensity not exceeding 4 mA and a maximum duration of 30 minutes. Compared to taVNS, tDCS typically requires longer stimulation time to achieve a therapeutic effect.

In some implementations, the electrical stimulation control module 1104 may realize different stimulation modes by controlling the switching timing of these three current paths. In monophasic stimulation mode, the electrical stimulation control module 1104 may selectively output combinations of anodic stimulation and charge release, or combinations of cathodic stimulation and charge release. While in biphasic stimulation mode, the electrical stimulation control module 1104 sequentially outputs anodic stimulation, cathodic stimulation, and charge release to ensure charge balance of stimulation. Particularly, during the acute phase of stroke patients, monophasic stimulation mode is prioritized, and after entering the subacute phase, gradual transition to biphasic stimulation mode is made based on the patient's tolerance level.

In some implementations, after aVNS stimulation is completed, the electrical stimulation control module 1104 enters a preset rest period (e.g., 2-5 minutes) to prevent cumulative stimulation effects. After the rest period ends, tDCS stimulation is initiated, at which time the electrical stimulation control module 1104 controls the current intensity and direction between the center electrode 151 (which may serve as anode) and ring electrode 152 (which may serve as cathode) of the concentric electrode 15 to generate the required direct current field. This timing control design not only prevents interference between the two stimulation modes but also ensures that each stimulation mode can achieve an optimal therapeutic effect.

In some implementations, the transceiver component 112 in the body 11 of the feedback-based neural electrical stimulation device may include a transmitter 1121, a receiver 1122, an encoder 1123, and a decoder 1124. Digital data may be converted into packets of specific format by the encoder 1123, then converted into wireless signals by the transmitter 1121 and transmitted, via antenna, for reception by corresponding receivers within range. Conversely, encoded commands and parameters transmitted by corresponding transmitters within range may be received by the antenna of the receiver 1122, then decoded into digital information by the decoder 1124 and sent to the data processing module 1103.

In some implementations, the charging component 113 in the body 11 of the feedback-based neural electrical stimulation device may include a charging interface 1131, a rechargeable battery 1132, a voltage regulator 1133, a buck-boost converter 1134, and a reference voltage 1135. The charging interface 1131 may be used to deliver power to the rechargeable battery 1132 for charging, and the voltage regulator 1133 may be used to convert battery voltage and interface voltage to system voltage, thus ensuring the system current load is within a specification range while minimizing voltage fluctuations that are caused by load variations and power coupling.

In some implementations, the voltage regulator 1133 may be further divided into digital and analog parts, mainly used to reduce interference from digital signals through power supply to analog circuits, and may separately activate digital and analog circuits to achieve reduced power consumption in certain operating modes. Since the target tissue for stimulation has capacitive properties, stimulation output usually requires higher voltage to maintain stimulation values, therefore the buck-boost converter 1134 may be used to boost the system voltage output from the voltage regulator 1133 to the voltage required by the electrical stimulator 150.

In some implementations, to ensure normal voltage output, the output of the buck-boost converter 1134 may undergo voltage detection through voltage division by the analog-to-digital conversion module 1102 to ensure normal high voltage output. The reference voltage 1135 may, after being electrically connected to the rechargeable battery 1132 or voltage regulator 1133, be used to provide a fixed voltage that has lower voltage, process, and temperature variation effects compared to system voltage, for use by the voltage regulator 1133 and other modules requiring voltage levels in the system.

In some implementations, the electrical stimulation control module 1104 automatically may adjust electrical stimulation modes based on physiological states. The data processing module 1103 analyzes PPG and EDA signal results, and when the user is in an anxious state, the electrical stimulation control module 1104 may adjust stimulation frequency to 20-250 Hz and use short pulses to activate the parasympathetic nervous system.

In some implementations, the feedback-based neural electrical stimulation device 1 may feature multiple safety protection mechanisms. The electrical stimulation control module 1104 has safety thresholds, monitors electrode impedance values and physiological signal changes, has overcurrent protection mechanisms, and has emergency stop functions that are installed on both the body 11 and mobile control device 2. Automatic calibration checks of electrode connection status are performed before each treatment.

In some implementations, the feedback-based neural electrical stimulation device 1 may integrate taVNS and tDCS stimulation modes. In some implementations, when applied to stroke patients and during the acute phase (e.g., within 2 weeks after onset), milder parameters are used: taVNS frequency of 20-100 Hz and tDCS current intensity of 0.5-1 mA. While during the subacute phase (e.g., 2 weeks to 3 months after onset), parameters may be increased to taVNS frequency of 100-250 Hz and tDCS current intensity of 1-2 mA.

In some implementations, the electrical stimulation control module 1104 may adopt a timing control strategy. A taVNS stimulation is performed first, followed by a tDCS stimulation after a charge releases. The interval time during the acute phase is 5-10 minutes, which may be shortened to 2-5 minutes during the subacute phase. Each parameter adjustment adopts gradual changes, with a minimum step value of 0.5 Hz for a taVNS frequency and intervals no less than 30 seconds, and a minimum step value of 0.1 mA for a current intensity and intervals no less than 1 minute.

In some implementations, the electrical stimulation control module 1104 may include a digital-to-analog converter 1141 and a switch module 1142. The switch module 1142 may realize three current paths: anodic stimulation, cathodic stimulation, and charge release, and may output monophasic or biphasic stimulation waveforms. When performing a tDCS stimulation, three-stage control may be adopted: ramp-up period (e.g., 15-30 seconds), sustained period (e.g., 10-30 minutes), and ramp-down period (e.g., 15-30 seconds).

In some implementations, the electrical stimulation control module 1104 may adopt different control strategies according to treatment phases: conservative parameter adjustment in the initial phase, flexible adjustment based on physiological signals in the stable phase, and gradual reduction strategy in the ending phase. If signal abnormality is detected, parameter adjustment may be temporarily frozen until the signal returns to stability.

In some implementations, the feedback-based neural electrical stimulation device 1 may establish customized treatment plans. The digital controller 110 may record information, such as treatment parameter settings, adjustment processes, and effect evaluations, transmit the information to the mobile control device 2 through the wireless transmission module 120 and upload the information to the server device 3 for analysis. The server device 3 analyzes users' response patterns to different stimulation parameters, identifies optimal parameter combinations, and returns them to the mobile control device 2 for reference.

In some implementations, the mobile control device 2 may provide a graphical interface and artificial intelligence platform that may display real-time physiological indicator changes, adjust treatment parameters, and provide personalized recommendations based on analytical results from the server device 3. The treatment records established by the mobile control device 2 include information, such as user data, treatment objectives, parameter settings, and physiological signal trends, which may be used for analysis and optimization by the artificial intelligence platform.

In some implementations, the server device 3 may establish treatment parameter models for different application scenarios based on large amounts of user data. The server device 3 establishes specialized parameter adjustment strategies based on factors, such as symptom type and age group, recommends suitable initial parameters for new users, and enables the feedback-based neural electrical stimulation device 1 to precisely meet different users' needs. The feedback-based neural electrical stimulation device 1 may also automatically adjust treatment schedules based on users' daily routines and physiological state changes.

In some implementations, the feedback-based neural electrical stimulation device 1 may be primarily applied to stroke auxiliary treatment. During the acute phase, the feedback-based neural electrical stimulation device 1 regulates the autonomic nervous system through taVNS combined with tDCS to promote neuroplasticity While in the subacute phase, parameters are adjusted according to recovery progress. The data processing module 1103 and the artificial intelligence platform of the mobile control device 2 may analyze PPG and EDA signals to monitor neural function recovery, based on which the electrical stimulation control module 1104 dynamically adjusts stimulation parameters.

In some implementations, the feedback-based neural electrical stimulation device 1 may also be used for other clinical applications. In emotional disorder treatment, parameters may be adjusted based on physiological signals. For example, increasing frequency to 20-250 Hz during anxiety to activate the parasympathetic nervous system. In chronic pain management, pain relief mechanisms may be activated by adjusting taVNS parameters, and pain-related brain regions may be modulated using tDCS. In daily health management, the feedback-based neural electrical stimulation device 1 may be used for stress management, sleep quality improvement, and attention enhancement.

The implementations described above are only used to illustrate the technical solutions of the present disclosure and are not intended to be limiting; although the present disclosure has been described in detail with reference to the preceding implementations, one with ordinary skill in the art should understand that: they may still modify the technical solutions described in the preceding implementations, or make equivalent substitutions for some technical features thereof; and such modifications or substitutions do not cause the corresponding technical solutions to depart from the scope of the technical solutions of the implementations of the present disclosure.

What is claimed is:

1. A feedback-based neural electrical stimulation system comprising a wearable feedback-based neural electrical stimulation device, wherein the feedback-based neural electrical stimulation device comprises:
a sensing module, for measuring physiological feedback signals from a user, comprising:
a photoplethysmography (PPG) sensor for measuring PPG signals; and
an electrodermal activity (EDA) sensor for measuring EDA signals;
an electrical stimulator, for providing electrical stimulation to the user, comprising:
a tragus electrode configured to attach to a tragus of the user for providing transcutaneous auricular vagus nerve stimulation (taVNS); and
a concentric electrode comprising a center electrode and a ring electrode, the concentric electrode configured to attach to skin of the user for providing transcranial direct current stimulation (tDCS); and
a digital controller electrically connected to both the sensing module and the electrical stimulator for receiving the physiological feedback signals from the sensing module and outputting electrical stimulation control signals to the electrical stimulator so as to control the electrical stimulation.

2. The feedback-based neural electrical stimulation system of claim 1, wherein the digital controller receives the PPG signals from the sensing module and converts the PPG signals to frequency domain for obtaining heart rate variability, analyzes high-frequency components and low-frequency components of the heart rate variability, determines an activity level of a parasympathetic nerve based on an energy ratio between the high-frequency components and the low-frequency components, and adjusts the electrical stimulation control signals based on the control of the electrical stimulation of the electrical stimulator.

3. The feedback-based neural electrical stimulation system of claim 2, wherein the high-frequency components are in a frequency range of 0.15 to 0.40 Hz, and the low-frequency components are in a frequency range of 0.06 to 0.15 Hz.

4. The feedback-based neural electrical stimulation system of claim 1, wherein the digital controller receives the EDA signals from the sensing module and decomposes the EDA signals into a skin conductance level (SCL) component and a skin conductance response (SCR) component, determines an activity level of a sympathetic nerve based on the SCL component, and adjusts the electrical stimulation control signals based on the control of the electrical stimulation of the electrical stimulator.

5. The feedback-based neural electrical stimulation system of claim 1, wherein the digital controller determines an activity level of a parasympathetic nerve based on the PPG signals and determines an activity level of a sympathetic nerve based on the EDA signals, and adjusts the electrical stimulation control signals to control a stimulation frequency and a pulse width of the electrical stimulation based on the activity level of the parasympathetic nerve and the activity level of the sympathetic nerve.

6. The feedback-based neural electrical stimulation system of claim 1, further comprising:
a mobile control device, for wireless communication with the wearable feedback-based neural electrical stimulation device, configured to display the physiological feedback signals and the electrical stimulation control signals and provide control commands to adjust the electrical stimulation control signals; and
a server device, for wireless communication with the wearable feedback-based neural electrical stimulation device and the mobile control device, configured to store and analyze the physiological feedback signals and the electrical stimulation control signals.

7. The feedback-based neural electrical stimulation system of claim 6, wherein the wearable feedback-based neural electrical stimulation device further comprises:
a wireless transmission module, for wireless communication with the mobile control device and the server device, electrically connected to the digital controller to transmit the physiological feedback signals, the electrical stimulation control signals, and the control commands.

8. The feedback-based neural electrical stimulation system of claim 1, wherein the wearable feedback-based neural electrical stimulation device further comprises:
a body configured to accommodate the digital controller with the tragus electrode and the concentric electrode disposed on a surface of the body;
an earplug element coupled to the body for securing the wearable feedback-based neural electrical stimulation device to the ear of the user; and
a sensing element disposed on a surface of the earplug element to contact the ear of the user and configured to accommodate the PPG sensor and the EDA sensor.

9. The feedback-based neural electrical stimulation system of claim 8, wherein the tragus electrode is a contact electrode or a snap-fit electrode.

10. The feedback-based neural electrical stimulation system of claim 9, wherein the contact electrode is a direct contact electrode or a spring-mechanism electrode.

11. The feedback-based neural electrical stimulation system of claim 9, wherein the snap-fit electrode comprises a male snap and a female snap that are detachably assembled with each other, one of the male snap and the female snap contacts the ear of the user, and the other is disposed on the body.
